(19) United States
(12) Patent Application Publication
(10) Pub. No.: US 2019/0159992 A1
(43) Pub. Date: May 30, 2019

(54) Hydrophobically-Modified Chitosan for Use in Cosmetics and Personal Care Applications

(71) Applicant: GEL-E, Inc., College Park, MD (US)

(72) Inventor: Matthew Dowling, College Park, MD (US)

(21) Appl. No.: 16/091,807

(22) PCT Filed: Apr. 6, 2017
(86) PCT No.: PCT/US17/26389

§ 371 Date: Oct. 5, 2018

Related U.S. Application Data
(60) Provisional application No. 62/319,265, filed on Apr. 6, 2016.

(51) Int. Cl. A61K 8/73; A61Q 19/08; A61Q 19/10; A61Q 19/00; A61Q 1/02; A61Q 1/10; A61Q 5/02; A61Q 5/12; A61Q 15/00

(52) U.S. Cl. CPC A61K 8/736; A61K 8/733; A61K 8/731; A61Q 19/08; A61Q 19/10; A61K 2800/54; A61Q 1/02; A61Q 1/10; A61Q 5/02; A61Q 5/12; A61Q 15/00; A61Q 19/007

(57) ABSTRACT

A cosmetic article that includes a hydrophobically-modified biopolymer and a cosmetic application. The cosmetic application is selected from the group consisting of mascara, moisturizing creams, moisturizing lotions, facial cleansers, wrinkle-reducing gels/creams/lotions, shampoos, conditioners, soaps, deodorants, acne treatment, thy-skin treatment, blemish concealers, coloring make-up, and controlled molecular release matrices for fragrances.

### CROSS-REFERENCE TO RELATED APPLICATIONS

This application claims the priority benefit under 35 U.S.C. § 119(e) of U.S. Provisional Application No. 62/319,265, entitled “HYDROPHOBICALLY-MODIFIED CHITOSAN FOR USE IN COSMETICS AND PERSONAL CARE APPLICATIONS” filed Apr. 6, 2016, which is incorporated herein by reference in its entirety.

### FIELD OF THE INVENTION

This invention relates to the field of cosmetic products, more specifically skin compositions containing hydrophobically-modified chitosan.

### BACKGROUND

Cosmetic products represent a large and growing worldwide market. The continuous development of new active ingredients for cosmetics and personal care products is one of the most important areas of research in this industry. As a result, there are a significant number of novel cosmetic products that are based on this new generation of active ingredients.

For cosmetics and personal care compositions, materials that provide optimal adherence to skin, have a water repellant nature, and which are easy to remove, are desirable. Further, materials that provide other advantages with routine use such as antimicrobial activity and improvements in skin health and appearance are desirable. Personal care compositions that assist in alleviating or attenuating the symptoms associated with skin disorders such as psoriasis, eczema, dry skin, pruritis, sun burns, impetigo, aging skin, are also desirable.

The present invention addresses these and other objectives.

### SUMMARY

It is one object of the present invention to provide a cosmetic or personal care article, comprising a hydrophobically-modified biopolymer, and a composition suitable for use as a cosmetic or for personal care. In some preferred embodiments, the cosmetic or personal care composition is selected from the group consisting of mascara, moisturizing creams, moisturizing lotions, facial cleansers, wrinkle-reducing gels/creams/lotions, shampoos, conditioners, soaps, deodorants, acne treatment, dry-skin treatment, blemish concealers, coloring make-up, and controlled molecular release matrices for fragrances. In various embodiments, routine use of the compositions described herein provide for improvements in skin health and/or appearance.

The cosmetic or personal care article includes a hydrophobically-modified biopolymer, which may be selected from hydrophobically-modified chitosan, hydrophobically-modified alginate, and hydrophobically-modified cellulosic. The hydrophobically-modified biopolymer, in some embodiments, comprises a plurality of hydrophobic substituents covalently attached to the polymer and wherein the hydrophobic substituents comprise hydrocarbon groups, including linear, branched, or cyclic hydrocarbons. The polymer backbones may range from 25,000 to 1,500,000 grams per more, with hydrophobic substituents present at from 1 to 100 moles per mole of biopolymer. For example, the hydrophobic substituent may occupy up to 50% of available functional groups of the biopolymer. In some embodiments, the cosmetic or personal care article has a hydrophobically modified biopolymer in a concentration of about 0.1% to about 5% by weight. In another embodiment, the concentration of biopolymer is about 2.0% to about 4% by weight.

In a further embodiment, the hydrophobically-modified chitosan is selected from the group consisting of chitosan salts: chitosan lactate, chitosan salicylate, chitosan pyrrolidone carboxylate, chitosan itaconate, chitosan niacinate, chitosan formate, chitosan acetate, chitosan gallate, chitosan glutamate, chitosan maleate, chitosan aspartate, chitosan glycolate and quaternary amine substituted chitosan and salts thereof. In another preferred embodiment, the hydrophobically-modified alginate is selected from the group consisting of sodium alginate, potassium alginate, magnesium alginate, calcium alginate, and aluminum alginate. In yet a further embodiment, the hydrophobically-modified cellulosic is selected from the group consisting of hydroxyethyl cellulose, hydroxypropyl cellulose, methyl cellulose, hydroxypropyl methyl cellulose, and hydroethyl methyl cellulose.

A method, in accordance with one embodiment of the present invention, comprises the step of applying the cosmetic or personal care composition to the skin to improve skin health and/or appearance. For example, in some embodiments, the composition provides for wrinkle-reduction. In some embodiments, the composition reduces microbial burden to a healthy level, which can be of particular importance for individuals prone to acne, infection, or atopic dermatitis. In various embodiments, the composition is applied routinely, such as about daily. The hydrophobically-modified biopolymer solution may be applied as one of a liquid spray, cream, lotion, gel, or foam.

### DESCRIPTION OF THE FIGURES

FIG. 1 shows the antibacterial activity of hydrophobically-modified chitosan in a bacterial clearing test. 10 μl of 0.5% hydrophobically-modified chitosan solution produce clearing zones up to 10 mm in diameter.

FIG. 2 compares the antimicrobial properties of chitosan and hydrophobically-modified chitosan, alongside ampicillin, against Methicillin-resistant Staphylococcus aureus (MRSA). Hm-chitosan at 0.5 wt % achieves a log killing of >2, whereas native chitosan (0.5 wt %) achieves a log killing of ˜1. In contrast, ampicillin at high dose (100 μg/ml) achieves only ˜0.5 log killing.

### DETAILED DESCRIPTION

Reference will now be made in detail to the presently preferred embodiments of the invention, examples of which are illustrated in the accompanying drawings.

In various aspects of the invention, a cosmetic or personal care article comprises a hydrophobically-modified (hm) biopolymer and a composition suitable for use as a cosmetic or for personal care. The hm-biopolymer can be a polysaccharide and may have physical and/or biological properties advantageous for routine application to skin or hair. As utilized herein, the term “cosmetic or personal care application” means any cosmetic or personal care product such as mascara, moisturizing creams, moisturizing lotions, facial cleansers, wrinkle-reducing gels/creams/lotions, shampoos, conditioners, soaps, deodorants, acne treatment, dry-skin treatment, blemish concealers, other standard coloring make-up, and controlled molecular release matrices for fragrance and medication.

In preferred embodiments, the current invention provides a hydrophobically-modified polymer matrix capable of interactions with skin and acting as a tensioning polymer in a wrinkle-reduction cream/lotion wherein the hydrophobically-modified polysaccharide is applied about 0.1% to about 2.5% by weight relative to the total weight of the composition of the biopolymer. By “tensioning polymer”, it is meant, a film forming polymer that is capable of adhering to and exerting a tensioning force upon a substrate. The term “about,” as used herein when referring to a measurable value such as an amount of a compound, dose, time, temperature, and the like, is meant to encompass variations of up to 10% variability of the specified amount.

In another embodiment of this invention, the hydrophobically-modified polysaccharides act as a thickening agent for cosmetic compositions such as mascara, moisturizing creams, moisturizing lotions, facial cleansers, shampoos, conditioners, soaps, deodorants, acne treatment, dry-skin treatment, blemish concealers, or other standard coloring make-up. In this embodiment, hydrophobically-modified polysaccharide is applied about 0.1% to about 2.5% by weight relative to the total weight of the composition of the biopolymer.

The polymer that forms the backbone of hydrophobically-modified polysaccharide, such as chitosan, is of synthetic or natural origin, including for example, water-soluble polysaccharides and water-soluble polypeptides. In particularly preferred embodiments, the polymer is one or more hydrophobically-modified polysaccharides, including but not limited to cellulosics, chitosans and alginates, all of which are abundant, natural biopolymers. One advantage of these types of materials is that they allow for the transfer of oxygen and moisture required for cosmetic applications.

The natural origin of these polysaccharides varies, cellulosics are found in plants, whereas chitosans and alginates are found in the exoskeleton or outer membrane of a variety of living organisms. Many of these naturally occurring polymers, in addition to being able to form long stable chains for forming the backbone of the current invention, have other benefits that may promote further advantages for their use in cosmetic applications. For instance, chitosan also has inherent anti-microbial properties; this is important for making cosmetic compositions last longer, and with routine use can provide a positive impact on the skin microbial burden. Positive charges along the backbone of chitosan cause it to interact electrostatically with negatively tissues such as skin which makes it very adhesive and beneficial to cosmetic compositions.

The form of the natural polymers used may vary to include standard states, derivatives and other various formulations. For example, the hm-cellulosics may be prepared from, without limitation, hydroxyethyl cellulose, hydroxypropyl cellulose, methyl cellulose, hydroxypropyl methyl cellulose, and/or hydroethyl methyl cellulose. Hm-Chitosans may be prepared from, without limitation, the following chitosan salts: chitosan lactate, chitosan salicylate, chitosan pyrrolidone carboxylate, chitosan itaconate, chitosan niacinate, chitosan formate, chitosan acetate, chitosan gallate, chitosan glutamate, chitosan maleate, chitosan aspartate, chitosan glycolate and quaternary amine substituted chitosan and salts thereof. Hm-Alginates may be prepared from, without limitation, sodium alginate, potassium alginate, magnesium alginate, calcium alginate, and/or aluminum alginate. It is to be understood that various other forms of any of these natural polysaccharides that provide the proper functional capabilities may be employed without departing from the scope and spirit of the present invention.

In some embodiments of this invention the polymeric component of the current invention is a mixture of polysaccharides. For instance, the mixture may be of various different sub-classes of a single polymer class. Alternatively, the mixture may include two or more different classes of polymer, for instance a cellolusic and a chitosan.

In a preferred embodiment, a matrix of the current invention is formed through the binding of numerous hydrophobically-modified chitosan compounds. These novel compounds consist of a biopolymer (e.g., chitosan) backbone that includes a hydrophilically reactive functional group (e.g., amino groups) that binds with the hydrophilically reactive head groups (e.g., carbonyl functional group) of an amphiphilic compound (e.g., aldehyde). The head group is further associated with a hydrophobic tail group. In the current embodiment, the hydrophobic tail may be for example a hydrocarbon. Thus, a hydrophobic tail is associated with the biopolymer's chitosan backbone providing the hydrophobic modification to the molecule that extends from the backbone and may interact with a surrounding environment in numerous ways, such as through hydrophobic interaction with other tissues, cells, molecules and/or structures. Without being bound by theory, the hydrophobic interaction between the modified chitosan and the bilayer of various tissues and/or cells may occur via the “insertion and anchoring” of the hydrophobic tail group of the short hydrophobic substituent into the bilayer membrane of the tissues or cells. The insertion process is driven by the generally understood hydrophobic interaction and those forces that are at work which tend to group like molecules when they exist in a heterogeneous environment. Thus, the hydrophobic effect or interaction is evidenced by the tendency of hydrophobic components to group together versus interacting or bonding with other molecules.

One exemplary embodiment of such hm-biopolymer is modified Chitosan. Chitosan is the common name of the linear, random copolymer that consists of β-(1-4)-linked D-glucosamine and N-acetyl-D-glucosamine. The molecular structure of chitosan consists of a linear backbone linked with glycosidic bonds. Chitosan is the major component of crustacean shells such as crab, shrimp, krill and crawfish shells. Additionally, chitosan is the second most abundant natural biopolymer after cellulose. Commercial chitosan samples are typically prepared by chemical de-N-acetylation of chitin under alkaline conditions. Depending on the source of the natural chitin (extracted from shells) and its production process, chitosan can differ in size (average molecular weight Mw) and degree of N-acetylation (% DA). Although the poor solubility of chitosan in water and in common organic solvents limits some of its applications, the reactive amino groups in the chitosan backbone make it possible to chemically conjugate chitosan with various biological molecules and to improve its utilization in cosmetics applications.

Chitosan is a deacetylated derivative of chitin with a degree of % DA that may range between 40 to 100%, or in some embodiments 60 to 100%, which determines the charge density. Chitosan is a linear polysaccharide composed of repeating -(1-4)-linked D-glucosamine monomeric units, its deacetylated structure is shown in Formula 1 below.

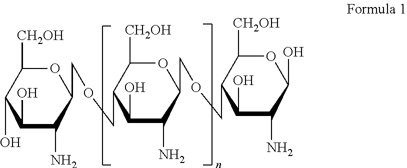

Chitosan structure showing three of the repeating beta-(1-4)-linked D-glucosamine units (deacetylated)

These repeating monomeric units include a free amino group (functional group) and may make molecules or compounds containing chitosan or its derivatives readily reactive. The hydrophobic modification of the chitosan backbone is through the association of an amphiphilic compound with the amino group, such that the hydrophobic tail of the amphiphilic compound is bound with the hydrophilic backbone structure.

Hydrophobically-modified (hm) chitosan is a derivative of chitosan which has much broader amphiphilic properties when compared with the parent biopolymer, thus expanding its utility in various applications. Here, different preparation conditions, which result in N-alkylated or N-acylated chitosans, a diverse degree of substitution and hydrophobicity are discussed. Hm-chitosan has moisture-retention and absorption properties, anti-microbial properties, anti-oxidant properties, delivery properties and emulsion stabilization properties which make it useful for a variety of cosmetic applications. These applications include mascara, moisturizing creams, moisturizing lotions, facial cleansers, wrinkle-reducing gels/creams/lotions, shampoos, conditioners, soaps, deodorants, acne treatment, dry-skin treatment, blemish concealers, other standard coloring make-up, and controlled molecular release matrices for fragrance and medication. Chitosan is a uniquely robust, durable material which is able to be formulated into a variety of product form factors. The addition of hydrophobic moieties to the backbone of chitosan increases its capability to serve many different needs in the field of cosmetics, taking the form of sponges, powders, fibers, gels, films, foams, creams, lotions, putties, and liquids.

Typically, and for the purposes of the preferred embodiments of the instant application, these hydrophobically-modified polymers (biopolymers) are referenced as being composed of a chitosan “backbone”, “scaffold”, and/or “lattice”. Thus, the backbone of the hydrophobically-modified biopolymer film matrix of the preferred embodiments of the current invention is the biopolymer chitosan. Other biopolymers, including but not limited to the cellulosics and alginates, which include similar characteristics of the chitosan backbone may be employed with departing from the scope and spirit of the instant invention.

The wrinkle-reduction cream/lotion and thickening agent of certain embodiments include at least one polymer and a plurality of hydrophobic substituent attached along the backbone of the polymer. The hydrophobic substituent preferably includes a hydrocarbon group having linear, branched, or cyclic hydrocarbons of from 4 to 100 carbons in length. In some embodiments, the hydrocarbons have from about 8 to about 18 carbon atoms attached to the backbone of the at least one polymer.

Alginates can be hydrophobically-modified by exchanging their positively charged counterions (e.g. Na+) with tertiary-butyl ammonium (TBA*) ions using a sulfonated ion exchange resin. The resulting TBA-alginate is dissolved in dimethylsulfoxide (DMSO) where reaction occurs between alkyl (or aryl) bromides and the carboxylate groups along the alginate backbone. Alginate can also be modified by fatty amine groups (e.g. dodecyl amine), followed by addition of 1-ethyl-3-(3-dimethylaminopropyl)carbodiimide, via EDC coupling.

Cellulosics can be hydrophobically-modified by first treating the cellulosic material with a large excess highly basic aqueous solution (e.g. 20 wt % sodium hydroxide in water). The alkali cellulose is then removed from solution and vigorously mixed with an emulsifying solution (for example, oleic acid) containing the reactant, which is an alkyl (or aryl) halide (e.g. dodecyl bromide).

Chitosans can be hydrophobically-modified by reaction of alkyl (or aryl) aldehydes with primary amine groups along the chitosan backbone in a 50/50 (v/v) % of aqueous 0.2 M acetic acid and ethanol. After reaction, the resulting Schiff bases, or imine groups, are reduced to stable secondary amines by dropwise addition of the reducing agent sodium cyanoborohydride.

The degree of substitution of the hydrophobic substituent on the polymer is up to 50% of available functional groups, for example, amines in the case of chitosan. For example, the hydrophobic substituent can be added to from 10 to 50% of available amines, or from 20 to 50% of available amines, or from 30 to 50% of available amines. It is contemplated that more than one particular hydrophobic substituent is substituted onto the polymer, provided that the total substitution level is substantially within the ranges set forth above.

In some embodiments, the hydrophobic substituent is derived from an amphiphilic compound, meaning it is composed of a hydrophilic Head group and a hydrophobic Tail group. The Head group binds with the polymer and positions the Tail group to extend from the backbone of the polymer scaffold. This makes the hydrophobic Tail group available for hydrophobic interactions. The Tail group is preferably a hydrocarbon of various forms. As used herein, hydrocarbon(s) are any organic molecule(s) or compound(s) with a “backbone” or “skeleton” consisting entirely of hydrogen and carbon atoms and which lack a functional group. Thus, these types of compounds are hydrophobic in nature, unable to react hydrophilically, and therefore provide an opportunity for hydrophobic interaction. The hydrophobic interaction capability of the amphiphilic compound bound to the chitosan backbone may provide significant advantage to the current invention when compared to the prior art in that the interaction of the hydrophobically-modified polymer matrix, whether chitosan, cellulosic or alginate based, with the bi-layer membrane softissue(s) and cell(s) is a self-driven, thermodynamic process requiring less energy input. Thus, regardless of any particular form of the Tail group of the short hydrophobic substituent (amphiphilic compound), so long as it provides the opportunity for hydrophobic interaction with the tissue(s), cell(s), or other hydrophobically active molecules and/or compounds it falls within the scope and spirit of the current invention.

Hydrocarbons that can be used in embodiments of this invention may be classified as saturated hydrocarbons, unsaturated hydrocarbons, and aromatic hydrocarbons. From this basic classification system there exist many derivatives and further types of compounds that build therefrom. For example, numerous and varied compounds include more than one aromatic ring and are generally referred to as polyaromatic hydrocarbons (PAH).

In some embodiments, that hydrophobic moiety or Tail is aliphatic. In aliphatic compounds, atoms can be joined together in straight chains, branched chains, or rings (in which case they are called alicyclic). They can be joined by single bonds (alkanes), double bonds (alkenes), or triple bonds (alkynes). Besides hydrogen, other elements can be bound to the carbon chain, the most common being oxygen, nitrogen, sulfur, and chlorine. Those of ordinary skill in the art will recognize that other molecules may also be bound to the carbon chains and that compounds of such heteroatomic structure are contemplated as falling within the scope of the current invention.

The hydrophobic Tail group of the amphiphilic compound bound to the polymer backbone of the current invention is capable of branching and/or allowing the inclusion of side chains onto its carbon backbone. This may promote the hydrophobic interaction between the hydrophobically-modified polymer matrix tissue, such as skin. It may be understood that the strength of the hydrophobic interaction is based upon the available amount of “hydrophobes” that may interact amongst themselves or one another. Thus, it may further promote the hydrophobic effect by increasing the amount of and/or “hydrophobic” nature of the hydrophobic Tail group that is interacting. For instance, a hydrophobic Tail group, which in its original form may include a hydrocarbon chain, may promote an increase in its hydrophobicity (ability to hydrophobically bond and strength of hydrophobic interaction) by having a hydrophobic side chain attach to one of the carbons of its carbon backbone. In a preferred embodiment of the current invention, this may include the attachment of various polycyclic compounds, which may include for instance various steroidal compounds and/or their derivatives such as sterol type compounds, more particularly cholesterol.

In other embodiments, the current invention contemplates the use of various molecules and/or compounds that may increase the hydrophobic interaction allowed between the Tail group and the bi-layer membrane of tissues and cells. It may also improve the one or more of antimicrobial activity, durability, water repellent properties, viscosity, and/or flexibility of the cosmetic product. The side chains may be linear chains, aromatic, aliphatic, cyclic, polycyclic, or any various other types of hydrophobic side chains as contemplated by those skilled in the art. Some of the contemplated hydrophobic side chains may include the following:

I. Linear Alkanes

| Number of C | | |
|---|---|---|
| Atoms | Formula | Common Name |
| 1 | CH₄ | Methane |
| 2 | C₂H₆ | Ethane |
| 3 | C₃H₈ | Propane |
| 4 | C₄H₁₀ | n-Butane |
| 5 | C₅H₁₂ | n-Pentane |
| 6 | C₆H₁₄ | n-Hexane |
| 7 | C₇H₁₆ | n-Heptane |
| 8 | C₈H₁₈ | n-Octane |
| 9 | C₉H₂₀ | n-Nonane |
| 10 | C₁₀H₂₂ | n-Decane |
| 11 | C₁₁H₂₄ | n-Undecane |
| 12 | C₁₂H₂₆ | n-Dodecane |
| 13 | C₁₃H₂₈ | n-Trideacane |
| 14 | C₁₄H₃₀ | n-Tetradecane |
| 15 | C₁₅H₃₂ | n-Pentadecane |
| 16 | C₁₆H₃₄ | n-Hexadecane |
| 17 | C₁₇H₃₆ | n-Heptadecane |
| 18 | C₁₈H₃₈ | n-Octadecane |
| 19 | C₁₉H₄₀ | n-Nonadecane |
| 20 | C₂₀H₄₂ | n-Eicosane |
| 21 | C₂₁H₄₄ | n-Heneicosane |
| 22 | C₂₂H₄₆ | n-Docosane |
| 23 | C₂₃H₄₈ | n-Tricosane |
| 24 | C₂₄H₅₀ | n-Tetracosane |
| 25 | C₂₅H₅₂ | n-Pentacosane |
| 26 | C₂₆H₅₄ | n-Hexacosane |
| 27 | C₂₇H₅₆ | n-Heptacosane |
| 28 | C₂₈H₅₈ | n-Octacosane |
| 29 | C₂₉H₆₀ | n-Nonacosane |
| 30 | C₃₀H₆₂ | n-Triacontane |
| 31 | C₃₁H₆₄ | n-Hentraiacontane |
| 32 | C₃₂H₆₆ | n-Dotriacontane |
| 33 | C₃₃H₆₈ | n-Tritriacontane |
| 34 | C₃₄H₇₀ | n-Tetratriacontane |
| 35 | C₃₅H₇₂ | n-Pentatriacontane |
| 36 | C₃₆H₇₄ | n-Hexatriacontane |
| 37 | C₃₇H₇₆ | n-Heptatriacontane |
| 38 | C₃₈H₇₈ | n-Octatriacontane |
| 39 | C₃₉H₈₀ | n-Nonactriacontane |
| 40 | C₄₀H₈₂ | n-Tetracontane |
| 41 | C₄₁H₈₄ | n-Hentatetracontane |
| 42 | C₄₂H₈₆ | n-Dotetracontane |
| 43 | C₄₃H₈₈ | n-Tritetracontane |
| 44 | C₄₄H₉₀ | n-Tetratetracontane |
| 45 | C₄₅H₉₂ | n-Pentatetracontane |
| 46 | C₄₆H₉₄ | n-Hexatetracontane |
| 47 | C₄₇H₉₆ | n-Heptatetracontane |
| 48 | C₄₈H₉₈ | n-Octatetracontane |
| 49 | C₄₉H₁₀₀ | n-Nonatetracontane |
| 50 | C₅₀H₁₀₂ | n-Pentacontane |
| 51 | C₅₁H₁₀₄ | n-Henpentacontane |
| 52 | C₅₂H₁₀₆ | n-Dopentacontane |
| 53 | C₅₃H₁₀₈ | n-Tripentacontane |
| 54 | C₅₄H₁₁₀ | n-Tetrapentacontane |
| 55 | C₅₅H₁₁₂ | n-Pentapentacontane |
| 56 | C₅₆H₁₁₄ | n-Hexapentacontane |
| 57 | C₅₇H₁₁₆ | n-Heptapentacontane |
| 58 | C₅₈H₁₁₈ | n-Octapentacontane |
| 59 | C₅₉H₁₂₀ | n-Nonapentacontane |
| 60 | C₆₀H₁₂₂ | n-Hexacontane |
| 61 | C₆₁H₁₂₄ | n-Henhexacontane |
| 62 | C₆₂H₁₂₆ | n-Dohexacontane |
| 63 | C₆₃H₁₂₈ | n-Trihexacontane |
| 64 | C₆₄H₁₃₀ | n-Tetrahexacontane |
| 65 | C₆₅H₁₃₂ | n-Pentahexacontane |
| 66 | C₆₆H₁₃₄ | n-Hexahexacontane |
| 67 | C₆₇H₁₃₆ | n-Heptahexacontane |
| 68 | C₆₈H₁₃₈ | n-Octahexacontane |
| 69 | C₆₉H₁₄₀ | n-Nonahexacontane |
| 70 | C₇₀H₁₄₂ | n-Heptacontane |
| 71 | C₇₁H₁₄₄ | n-Henheptacontane |
| 72 | C₇₂H₁₄₆ | n-Doheptacontane |
| 73 | C₇₃H₁₄₈ | n-Triheptacontane |
| 74 | C₇₄H₁₅₀ | n-Tetraheptacontane |
| 75 | C₇₅H₁₅₂ | n-Pentaheptacontane |
| 76 | C₇₆H₁₅₄ | n-Hexaheptacontane |
| 77 | C₇₇H₁₅₆ | n-Heptaheptacontane |
| 78 | C₇₈H₁₅₈ | n-Octaheptacontane |
| 79 | C₇₉H₁₆₀ | n-Nonaheptacontane |
| 80 | C₈₀H₁₆₂ | n-Otcacontane |
| 81 | C₈₁H₁₆₄ | n-Henoctacontane |
| 82 | C₈₂H₁₆₆ | n-Dooctacontane |
| 83 | C₈₃H₁₆₈ | n-Trioctacontane |
| 84 | C₈₄H₁₇₀ | n-Tetraoctacontane |
| 85 | C₈₅H₁₇₂ | n-Pentaoctacontane |
| 86 | C₈₆H₁₇₄ | n-Hexaoctacontane |
| 87 | C₈₇H₁₇₆ | n-Heptaoctacontane |
| 88 | C₈₈H₁₇₈ | n-Octaoctacontane |
| 89 | C₈₉H₁₈₀ | n-Nonaoctacontane |
| 90 | C₉₀H₁₈₂ | n-Nonacontane |
| 91 | C₉₁H₁₈₄ | n-Hennonacontane |
| 92 | C₉₂H₁₈₆ | n-Dononacontane |
| 93 | C₉₃H₁₈₈ | n-Trinonacontane |
| 94 | C₉₄H₁₉₀ | n-Tetranonacontane |
| 95 | C₉₅H₁₉₂ | n-Pentanonacontane |
| 96 | C₉₆H₁₉₄ | n-Hexanonacontane |
| 97 | C₉₇H₁₉₆ | n-Heptanonacontane |
| 98 | C₉₈H₁₉₈ | n-Octanonacontane |
| 99 | C₉₉H₂₀₀ | n-Nonanonacontane |
| 100 | C₁₀₀H₂₀₂ | n-Hectane |
| 101 | C₁₀₁H₂₀₄ | n-Henihectane |
| 102 | C₁₀₂H₂₀₆ | n-Dohectane |
| 103 | C₁₀₃H₂₀₈ | n-Trihectane |
| 104 | C₁₀₄H₂₁₀ | n-Tetrahectane |
| 105 | C₁₀₅H₂₁₂ | n-Pentahectane |
| 106 | C₁₀₆H₂₁₄ | n-Hexahectane |
| 107 | C₁₀₇H₂₁₆ | n-Heptahectane |
| 108 | C₁₀₈H₂₁₈ | n-Octahectane |
| 109 | C₁₀₉H₂₂₀ | n-Nonahectane |
| 110 | C₁₁₀H₂₂₂ | n-Decahectane |
| 111 | C₁₁₁H₂₂₄ | n-Undecahectane |

II. Cyclic Compounds
a. Alicyclic Compound/Cycloalkane/Cycloalkene: An organic compound that is both aliphatic and cyclic with or without side chains attached. Typically include one or more all-carbon rings (may be saturated or unsaturated), but NO aromatic character.
b. Aromatic hydrocarbon/Polycyclic aromatic hydrocarbon/Heterocyclic compound: Organic compounds with a ring structure containing atoms in addition to carbon, such as nitrogen, oxygen, sulfur, chloride, as part of the ring. May be simple aromatic rings, non-aromatic rings. Some examples are pyridine (C5H5N), Pyrimidine (C4H4N2) and Dioxane.

| | | Example |
|---|---|---|
| Polycyclic Compounds | Sub-Types | Compounds |
| Bridged Compound - | Bicyclo compound | adamantine |
| compounds which contain | | amantadine |
| interlocking rings | | biperiden |
| | | memantine |
| | | methenamine |
| | | rimantadine |
| Macrocyclic Compounds | Calixarene | |
| | Crown Compounds | |
| | Cyclodextrins | |
| | Cycloparaffins | |
| | Ethers, Cyclic | |
| | Lactans, macrocyclic | |
| | Macrolides | |
| | Peptides, Cyclic | |
| | Tetrapyrroles | |
| | Trichothecenes | |
| Polycyclic Hydrocarbons, | Acenaphthenes | |
| Aromatic. | Anthracenes | |
| | Azulenes | |
| | Benz(a)anthracenes | |
| | Benzocycloheptenes | |
| | Fluorenes | |
| | Indenes | |
| | Naphthalenes | |
| | Phenalenes | |
| | Phenanthrenes | |
| | Pyrenes | |
| | Spiro Compounds | |
| Steroids | Androstanes | |
| | Bile Acids and Salts | |
| | Bufanolides | |
| | Cardanolides | |
| | Cholanes | |
| | Choestanes | |
| | Cyclosteroids | |
| | Estranes | |
| | Gonanes | |
| | Homosteroids | |
| | Hydroxysteroids | |
| | Ketosteroids | |
| | Norsteroids | |
| | Prenanes | |
| | Secsteroids | |
| | Spirostans | |
| | Steroids, Brominated | |
| | Steroids, Chlorinated | |
| | Steroids, Fluorinated | |
| | Steroids, Heterocyclic | |

Without being bound by theory, the addition of the side chains may increase the stability and strength of the hydrophobic interaction between the tail group and other hydrophobically active locations, such as a hydrophobic cavity in the bi-layer membrane of various biological structures including tissue such as skin, This increase in strength and stability may provide further advantages in the ability of the hydrophobically-modified polymer matrix to self-assemble, such as adhering and providing a tensioning force to skin.

In various embodiments, the biopolymer is a hm-chitosan, which may be prepared from a chitosan having a degree of deacetylation of from about 40% to about 90%, such as from about 50% to about 80%, such as from about 60% to about 75%. In some embodiments, the degree of substitution of the hydrophobic substituent on the biopolymer is from about 1 to about 100 moles of the hydrophobic substituent per mole of the biopolymer. In some embodiments, the degree of substitution of the hydrophobic substituent on the polysaccharide is from about 40 to 65 moles of the hydrophobic substituent per mole of the polysaccharide. In some embodiments, the degree of substitution of the hydrophobic substituent on the polysaccharide is from about 1 to 30 moles of the hydrophobic substituent per mole of the polysaccharide. In some embodiments, the molecular weight of the polysaccharides used as the biopolymer range from about 25,000 to about 1,500,000 grams per mole.

In various embodiments, the molecular weight of the biopolymer ranges from about 40,000 to about 500,000 grams per mole, or from about 50,000 to about 250,000 grams per mole, or from about 50,000 to about 100,000 grams per mole. As used herein, the term “molecular weight” means weight average molecular weight. Methods for determining average molecular weight of bio-polymers include low angle laser light scattering (LLS) and Size Exclusion Chromatography (SEC). In performing low angle LLS, a dilute solution of the polysaccharide, typically 2% or less, is placed in the path of a monochromatic laser. Light scattered from the sample hits the detector, which is positioned at a low angle relative to the laser source. Fluctuation in scattered light over time is correlated with the average molecular weight of the polysaccharide in solution. In performing SEC measurements, again a dilute solution of biopolymer, typically 2% or less, is injected into a packed column. The polysaccharide is separated based on the size of the dissolved polymer molecules and compared with a series of standards to derive the molecular weight.

A hydrophobically-modified biopolymer material for incorporation into cosmetic products can be based on a solution of the hm-biopolymer that is about 0.1% to about 5.0% by weight relative to the total weight of the solution, or in some embodiments, about 0.5% to about 4%, or about 0.5% to about 3% of the total weight of the solution, or about 0.5% to about 2% of the total weight of the solution. In some embodiments, the solution is about 1.0% to about 5.0% by weight relative to the total weight of the solution of the biopolymer, or in some embodiments, about 1.5% to about 5%, or about 2.0% to about 4% of the total weight of the solution. In some embodiments, the hm-biopolymer solution is dried or lyophilized.

Hydrophobic moieties can be independently selected from saturated hydrocarbons (e.g., alkanes) and unsaturated hydrocarbons (e.g., alkenes, alkynes), which may be linear, branched or cyclic. In some embodiments, the hydrophobic moieties include aromatic hydrocarbons. In some embodiments, the hydrophobic moiety is a hydrocarbon having from about 4 to about 100 carbon atoms, or from about 8 to about 60 carbon atoms, or from about 8 to about 28 carbon atoms, or from about 8 to about 18 carbon atoms.

The hydrophobic substituents may be a hydrocarbon group having from about 8 to about 18 carbon atoms attached to the backbone of the one biopolymer, and in some embodiments comprises an alkyl group. In some embodiments, the hydrocarbon group comprises an arylalkyl group. As used herein, the term “arylalkyl group” means a group containing both aromatic and aliphatic structures.

The modified chitosan molecules exhibit various potential biological activities, such as antimicrobial, antifungal, antitumor and immunomodulatory activities. Hydrophobically-modified (hm) chitosan has utility in cosmetic and personal care products due to these properties, including for individuals exhibiting sings of acne, mild infection, or atopic dermatitis, which can be associated with and/or exacerbated by S. aureus “overgrowth”. Because of its broadly amphiphilic nature, hm-chitosan can be formulated into a large range of products which have utility in cosmetics and personal care, including mascara, wrinkle-reduction creams/lotions, deodorants, shampoos, soaps, blemish concealers, acne creams, moisturizers and fragrance carriers. Hm-chitosan is a stable, robust, and durable biopolymer which is capable of retaining its functionality for extremely long storage periods at room temperature.

The molecular weight of the polymers comprising the wrinkle-reduction cream/lotion ranges from about 50,000 to about 500,000 grams per gram mole, It is contemplated that the molecular weight of the polymers in the sponge or solution formulations may be less than or greater than the range identified without departing from the scope and spirit of the current invention, For instance, the molecular weight of the polymers comprising the thickening agent for cosmetic compositions from about 10,000 to about 200,000 grams per gram mole, As used herein, the term “molecular weight” means weight average molecular weight In preferred examples, average molecular weight of polymers is determined by low angle laser light scattering (LLS) and Size Exclusion Chromatography (SEC), In performing low angle LLS, a dilute solution of the polymer, typically 2% or less, is placed in the path of a monochromatic laser, Light scattered from the sample hits the detector, which is positioned at a low angle relative to the laser source, Fluctuation in scattered light over time is correlated with the average molecular weight of the polymer in solution, In performing SEC measurements, again a dilute solution of polymer, typically 2% or less, is injected into a packed column, The polymer is separated based on the size of the dissolved polymer molecules and compared with a series of standards to derive the molecular weight.

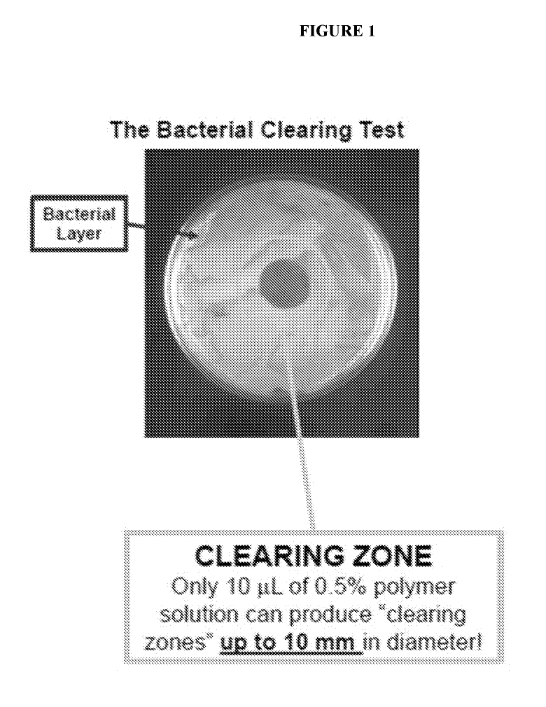

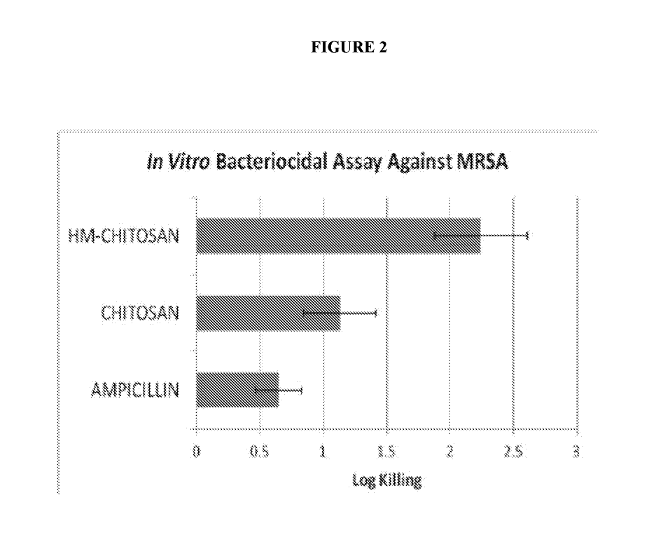

As discussed above, the hm-modified biopolymer, such as hm-chitosan, can have antimicrobial properties, including antibacterial and/or antifungal properties. In some embodiments, the hm-biopolymer can have antimicrobial properties against one or more common pathogens or odor-causing bacteria or fungus. Example include: Pseudomonas aeruginosa, Acinetobacter baumanni, Klebsiella pneumonia, Escherichia coli, Staphylococcus aureus and Enterococcus faecalis. In some embodiments, the hm-biopolymer has antimicrobial properties against Methicillin-resistant Staphylococcus aureus (MRSA) as shown in FIGS. 1 and 2, a common pathogen found on skin which is easily spread by contact with contaminated surfaces.

In still some embodiments, the hm-biopolymer is active against one or more of Staphylococcus sp., Pseudomonas sp., Enterococcus sp., Shigella sp., Listeria sp., Bacillus sp., Lactobacillus sp., Salmonella sp., and Vibrio sp. In some embodiments, the hm-polymer has antifungal activity against one or more of Aspergillus sp., Fusarium sp., and Candida sp. The particular biopolymer can be selected in accordance with the disclosure for the desired antibacterial and/or anti-fungal profile, which can depend on the application of the cosmetic product. In the case of chitosan, hm-chitosan can have antimicrobial properties greater than native chitosan. In some embodiments, the hm-polymer is chitosan modified with hydrophobic groups having from 8 to 28 carbon atoms. The hm-polymer can further be designed for the desired durability, flexibility, and/or water repellant nature of the resulting cosmetic product, based on, for example, biopolymer molecular weight, amount of available amines or other functional group, type and amount of hydrophobic moieties, and processing technique for the hydrophobically-modified biopolymer for use in cosmetic products. In some embodiments, a foaming agent is incorporated prior to drying to modulate the flexibility and/or feel of the resulting material.

In some embodiments, the hm-polymer is formed from a dehydrated solution or foam, which has the potential to alter characteristics such as flexibility and feel of the resulting cosmetic product.

In other embodiments, the invention provides methods for treating skin and/or hair, comprising applying the cosmetic or personal care composition described herein. In various embodiments, the invention improves the appearance and/or health of skin. For example, in various embodiments, the composition reduces the appearance of wrinkles and/or loose skin, such as around the mouth, around the eyes, or forehead. In some embodiments, the composition is formulated as a cream, hydrogel, or foam for application to the skin (either for the face or body), and which reduces the severity or frequency of acne. In still other embodiments, the composition is applied to an individual exhibiting signs of atopic dermatitis, which is associated with overgrowth of S. aureus and potentially other commensal microbes. In these embodiments, the composition reduces the severity of the condition. In yet further embodiments, the composition can be utilized to treat various skin conditions such as psoriasis, eczema, dry skin, pruritis, sun burns, and impetigo. The hm-biopolymer reduces microbial burden while enhancing skin barrier integrity when used on inflammatory skin lesions. Additionally, the hm-biopolymer's antimicrobial properties assist in preventing infections when used to treat such skin lesions.

In certain embodiments, the composition is applied routinely, such as about daily, or from about 1 to 5 times per week, and can be used for a prolonged period of time (e.g., one month or more, or six months or more).

Other aspects and embodiments of the invention will be apparent to the skilled artisan from this disclosure.

1. A cosmetic or personal care article, comprising:
a hydrophobically-modified biopolymer, and
a composition suitable for cosmetic or personal care.

2. The cosmetic article of claim 1, wherein the composition is selected from the group consisting of mascara, moisturizing creams, moisturizing lotions, facial cleansers, wrinkle-reducing gels/creams/lotions, shampoos, conditioners, soaps, deodorants, acne treatment, dry-skin treatment, blemish concealers, coloring make-up, and controlled molecular release matrices for fragrances.

3. The cosmetic article of claim 2, wherein the hydrophobically-modified biopolymer is selected from the group consisting of hydrophobically-modified chitosan, hydrophobically-modified alginate, and hydrophobically-modified cellulosic.

4. The cosmetic article of claim 3, wherein the hydrophobically-modified biopolymer comprises a plurality of hydrophobic substituents covalently attached to the polymer and wherein the hydrophobic substituents comprise linear, branched, or cyclic hydrocarbon groups, which optionally have from about 8 to about 18 carbon atoms.

5. The cosmetic article of claim 4, wherein the hydrophobic substituents on the biopolymer occupy up to 50% of available functional groups of the biopolymer.

6. The cosmetic article of claim 5, wherein the hydrophobically-modified biopolymer has a concentration of about 0.1% to about 2.5% by weight.

7. The cosmetic article of claim 3, wherein the hydrophobically-modified chitosan is selected from the group consisting of chitosan salts: chitosan lactate, chitosan salicylate, chitosan pyrrolidone carboxylate, chitosan itaconate, chitosan niacinate, chitosan formate, chitosan acetate, chitosan gallate, chitosan glutamate, chitosan maleate, chitosan aspartate, chitosan glycolate and quaternary amine substituted chitosan and salts thereof.

8. The cosmetic article of claim 3, wherein the hydrophobically-modified alginate is selected from the group consisting of sodium alginate, potassium alginate, magnesium alginate, calcium alginate, and aluminum alginate.

9. The cosmetic article of claim 3, wherein the hydrophobically-modified cellulosic is selected from the group consisting of hydroxyethyl cellulose, hydroxypropyl cellulose, methyl cellulose, hydroxypropyl methyl cellulose, and hydroethyl methyl cellulose.

10. The cosmetic article of claim 5, wherein the hydrophobically-modified biopolymer is present in a concentration of about 0.1% to about 5%% by weight.

11. A method, comprising:
applying the hydrophobically-modified biopolymer composition of any one of claims 1 to 10 to the skin.

12. The method of claim 11, wherein the composition is applied for wrinkle-reduction, acne reduction, or treatment of atopic dermatitis.

13. The method of claim 11 or 12, wherein the biopolymer is selected from the group consisting of chitosans, alginates, and cellulosics.

14. The method of any one of claims 11 to 13, wherein the hydrophobic moieties comprise 8 to 18 hydro-carbon residues.

15. The method of any one of claims 11 to 14, wherein the hydrophobically-modified biopolymer solution has a concentration of about 0.1% to about 2.5% by weight relative to the total weight of the solution of the biopolymer.

16. The method of any one of claims 11 to 15, wherein the hydrophobic moieties are covalently attached to as many as 50% of available amines of chitosan.

17. The method of any one of claims 11 to 16, wherein the hydrophobically-modified biopolymer composition is applied as one of a liquid spray, cream, lotion, gel, or foam.
